(19) United States
(12) Patent Application Publication
(10) Pub. No.: US 2018/0042751 A1
(43) Pub. Date: Feb. 15, 2018

(54) OPTIONAL TWO PIECE SUPRA MALLEOLAR AND ANKLE FOOT ORTHOSIS SYSTEM AND METHOD THEREFOR

(71) Applicant: JOSEPH A. DEROSE, SCOTTSDALE, AZ (US)

(72) Inventor: JOSEPH A. DEROSE, SCOTTSDALE, AZ (US)

(21) Appl. No.: 15/611,565

(22) Filed: Jun. 1, 2017

Related U.S. Application Data
(60) Provisional application No. 62/374,563, filed on Aug. 12, 2016.

(51) Int. Cl. A61F 5/01

(52) U.S. Cl. CPC A61F 5/0111; A61F 5/0127; A61F 5/0195

(57) ABSTRACT

An ankle and foot orthodic device has an insole. An ankle support is positioned under the insole and extending up above a foot and proximate an ankle of a user.

### RELATED APPLICATIONS

The present patent application claims the benefit of U.S. Provisional Application No. 62/374,563, filed Aug. 12, 2016, entitled “TWO PIECE SUPRA MALLEOLAR ORTHOSIS”, and which is incorporated herein by reference in its entirety

### TECHNICAL FIELD

The present application generally relates to orthotic devices, and, more particularly, to a two piece foot and ankle orthopedic device configured to avoid irritating the navicular tuberosity area and to exert more pressure either laterally to counteract the foot going in a supinatory movement or medially to counteract a pronatory movement.

### BACKGROUND

Foot and ankle orthopedic devices may be used to control the alignment and motions of the joints of the foot and ankle of a patient. The foot and ankle orthopedic device may be formed of multiple pieces, a foot piece and an ankle brace member. The foot piece may function similar to a foot orthosis. The foot piece may molded to conform to a bottom surface of the entire foot of the wearer. The foot portion may be used to reduce abnormal motion or abnormal position of the foot. The foot portion may further be used to control the abnormal motion or abnormal position of the lower extremity that is affected by the position and/or motion of the foot.

Two upright post members may extend up from the foot portion to form the ankle brace. The ankle brace may support the ankle, hold the foot and ankle in the correct position and correct foot drop.

Unfortunately, due to the length and rigidity of the upright post members forming the foot and ankle orthopedic device, the upright post members have a tendency to irritate the navicular tuberosity area of the wearer. Further, present foot ankle and foot orthopedic device do not allow the user to adjust the tension/pressure either laterally to counteract the foot going in a supinatory movement or medially to counteract a pronatory movement adjust.

Therefore, it would be desirable to provide a device and method that overcomes the above. The foot and ankle orthopedic device would be configured to avoid irritating the navicular tuberosity area. The foot and ankle orthopedic device would be able to adjust the pressure either laterally to counteract the foot going in a supinatory movement or medially to counteract a pronatory movement.

### SUMMARY

In accordance with one embodiment, an ankle and foot orthotic device is disclosed. The ankle and foot orthosis has an insole. An ankle support is positioned under the insole and extending up above a foot and proximate an ankle of a user.

In accordance with one embodiment, an ankle and foot orthotic device is disclosed. The ankle and foot orthotic device has an insole. An ankle support is positioned under the insole and extends up above a foot and proximate an ankle of a user. The ankle support has a “U” shaped member. An inner support member extends up and away from the bottom of the “U” shaped member. An outer support member extends up and away from the bottom of the “U” shaped member. The inner support member and the outer support member run from the proximal/plantar to distal/dorsal areas of the ankle. A pair of straps is provided, wherein an individual strap is coupled to opposing sides of the insole, wherein the pair of straps arranged in a crossing pattern and coupled to a respective one of the inner support member and outer support member to exert force to counteract movement of the foot.

### BRIEF DESCRIPTION OF THE DRAWINGS

The present application is further detailed with respect to the following drawings. These figures are not intended to limit the scope of the present invention but rather illustrate certain attributes thereof.

FIG. 1 is a perspective view of an exemplary foot and ankle orthotic device according to one aspect of the present application;

FIG. 2 is another perspective view of the exemplary foot and ankle orthotic device of FIG. 1 in accordance with one aspect of the present application;

FIG. 3 is an exploded perspective view of the exemplary foot and ankle orthotic device of FIG. 1 in accordance with one aspect of the present application;

FIG. 4 is a perspective view of an exemplary foot and ankle orthotic device according to one aspect of the present application;

FIG. 5 is another perspective view of the exemplary foot and ankle orthotic device of FIG. 4 in accordance with one aspect of the present application; and

FIG. 6 is an exploded perspective view of the exemplary foot and ankle orthotic device of FIG. 4 in accordance with one aspect of the present application.

### DESCRIPTION OF THE APPLICATION

The description set forth below in connection with the appended drawings is intended as a description of presently preferred embodiments of the disclosure and is not intended to represent the only forms in which the present disclosure can be constructed and/or utilized. The description sets forth the functions and the sequence of steps for constructing and operating the disclosure in connection with the illustrated embodiments. It is to be understood, however, that the same or equivalent functions and sequences can be accomplished by different embodiments that are also intended to be encompassed within the spirit and scope of this disclosure.

Embodiments of the exemplary foot and ankle orthopedic device may be configured to avoid irritating the navicular tuberosity area. The foot and ankle orthopedic device may be able to adjust the pressure either laterally to counteract the foot going in a supinatory movement or medially to counteract a pronatory movement.

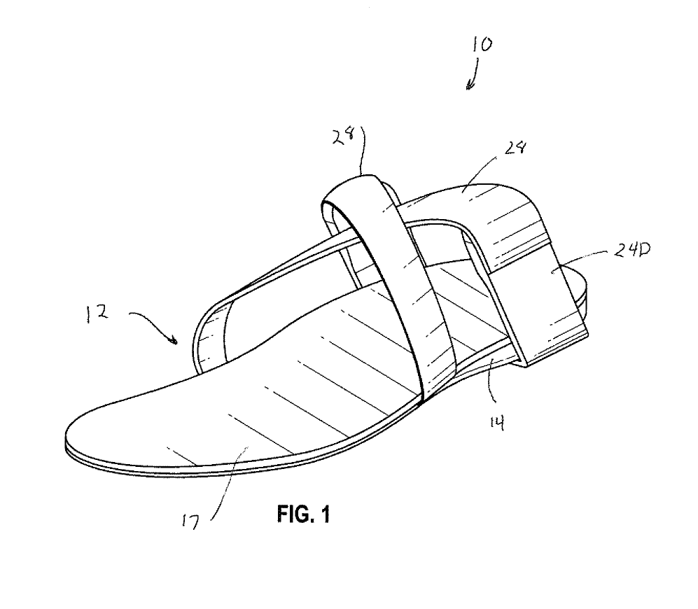

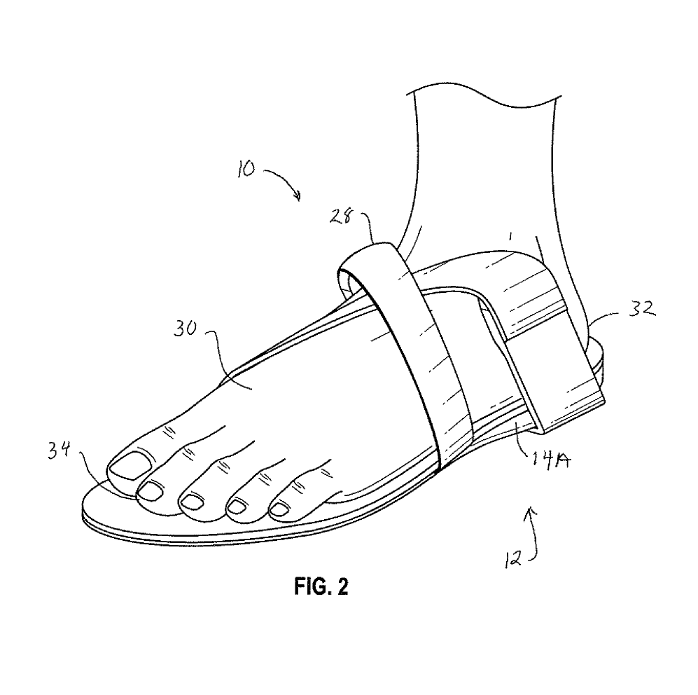

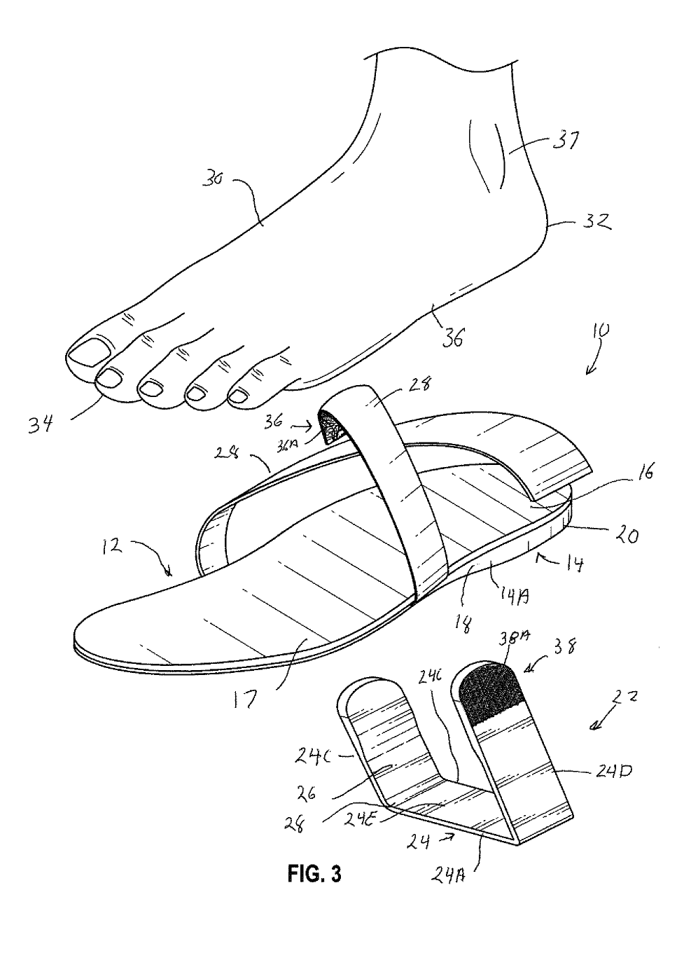

Referring now to FIG. 1-FIG. 3 a foot and ankle orthopedic device 10 (hereinafter device 10) may be shown. The device 10 may be formed of a customizable insole 12. The insole 12 may be customized to conform to a shape of a user's foot 30. A length of the insole 12 can be dependent on foot size and may extend from the user's heel 32 towards the front 34 of the foot 30.

The insole 12 may include a base layer 14 and an upper layer 16. The base layer 14 may extend from the back of the heel 32 of the user's foot past the arch 36 of the foot 30. The base layer 14 may be molded to conform to the bottom of the foot 30. The base layer 14 may be used to control the subtalar joint and foot biomechanics of the user. In general, the base layer 14 may be formed from a thin, firm material. For example, subortholen, polypropylene, copolymer, and carbon graphite composites may be used to form the base layer. The material forming the base layer 14 may be heated to allow the material to be malleable. Once heated, the base layer may be molded to conform to a shape of the bottom of the user's foot 30. Alternatively, the material forming the base layer 14 may be milled or formed via a 3D printer to conform to a shape of the bottom of the user's foot 30.

The base layer 14 may have a heel cup 16 formed on one end thereof. The heel cup 16 may be used to keep the heel 32 and hence the foot 30 from moving side to side. The heel cup 16 may further be used to elevate one side of the heel 32 to correct foot posture and gait. A medial longitudinal arch area 18 may be formed extending from the heel cup 16 along an inner side 14A of the base layer. The medial longitudinal arch area 18 may be used to support the arch 36 of the user's foot 30 and to redistribute the weight of the user to different areas to prevent excessive pressure. The medial longitudinal arch area 18 may also correct alignment issues.

A heel section 20 may be formed on a bottom section of the base layer 14 below the heel cup 16. The heel section 20 may slightly raise the area of the base layer 14 where the heel section is located.

The upper layer 17 may be applied over the base layer 14. The upper layer 17 may extend past the base layer 14 to the end of the user's foot 30. The upper layer 17 may be formed of a flexible material. A foam, gel or similar material may be used for the upper layer 17. The upper layer 17 may provide a cushion to relieve any soreness, discomfort or the like in the foot 30.

An ankle support 22 may be removably coupled to the insole 12. The ankle support 22 may be used to support the ankle 37 and foot 30 of the user. The ankle support 22 may control the position and motion of the ankle 37 due to injuries and/or irregularities of the ankle 37. Since the ankle support 22 is removable, this may allow the user to remove the ankle support 22 and just use the insole 12 of the device 10 as a foot orthosis.

In accordance with one embodiment, the ankle support 22 of the present embodiment may have support members both under and along the sides of the insole 12. The support members may be formed over both the insole 12 and the corrected cast of the patient's foot and lower leg.

The ankle support 22 may be formed of a “U” shaped member 24. The “U” shaped member 24 may be formed of a thin, semi-firm or firm material. The material used may be similar to that of the base layer 14. For example, subortholene, polypropylene, copolymer, and carbon graphite composites may be used to form the “U” shaped member 24. However, by having the material being thinner than that of the base layer 14, the ankle support 22 may have flexibility.

The bottom 24E of the “U” shaped member may be tapered from a front area 24A to a rear area 24B. The insole 12 may slide into the “U” shaped member 24 such that the heel section 20 rest within the “U” shaped member 24. The tapered configuration of the “U” shaped member 24 may allow the insole 12 to slide into the “U” shaped member 24 and prevents the heel cup 16 from running past the “U” shaped member 24.

The “U” shaped member 24 may have an inner support member 24C and an outer support member 24D. The inner support member 24B and an outer support member 24D extend up and away from the bottom 24E of the “U” shaped member 24. The inner support member 24C and an outer support member 24D may run from the proximal/plantar to distal/dorsal areas of the ankle. In accordance with one embodiment, the inner support member 24C may be of a different height than the outer support member 24D corresponding to a location of the ankle bone. The different profile of the inner support member 24C and the outer support member 24D serve multiple purposes. First, the angle and profile of the inner support member 24C may be configured to avoid the area of the navicular tuberosity. The navicular tuberosity may be a normally-occurring boney prominence on the inside of the foot 30 just above the arch 36. Prior art ankle support designs generally have support members which rub along this area and cause swelling, redness and pain. Thus, by configuring the inner support member 24C to avoid the area of the navicular tuberosity, the ankle support 22 may avoid an area of irritation and lessen the probability of swelling, redness and pain which may be caused by other ankle support devices. Second, the thinning or increased flexibility of the ankle support 22 over the malleoli (i.e., ankle bones), helps to diminish irritation of this area. Third, the profile of the inner support member 24C and the outer support member 24D may be less bulky and noticeable, thereby making it more aesthetically pleasing than prior art designs and may provide for a better shoe fit.

Padding 26 may be applied to an inner surface of the inner support member 24C and/or outer support member 24D. The padding 26 may be used to provide a more comfortable feel and to lessen irritation of the area upon which the inner support member 24C and/or outer support member 24D rest. A bottom interior area of inner support member 24C and/or outer support member 24D may have no padding 26. This may form a channel 28 in the bottom interior area of inner support member 24C and/or outer support member 24D. The channel 28 may provide a guide for sliding the insole 12 into the “U” shaped member 24.

The device 10 may have a pair of straps 28. Each strap 28 is attached to the insole 12. In accordance with one embodiment, a strap 28 is attached to opposing sides of the base layer 14 past the medial longitudinal arch area 18. The pair of straps 28 may be aligned across from each other. In accordance with one embodiment, a single strap 28 may be used. The base layer 14 may be positioned and attached to a middle area of the strap 28, opposing end members of the strap 28 forming the pair of straps 28 on opposing sides of the insole 12.

The straps 28 may be used to exert force to counteract movement of the foot 30. The straps 28 may be attached in a crossing pattern such that each strap 28 attaches to an opposing side of the ankle support 22. In accordance with one embodiment, the straps 28 may be attached in a crossing pattern to the inner support member 24C and outer support member 24D of the ankle support 22. The straps 28 may be attached in a crossing pattern over a top of the foot 30 and/or shoe of the user. The straps 28 may allow the user to exert more force either laterally, to counteract the foot 30 going in a supinatory movement outward and to the outside, or medially to counteract a pronatory movement inward and to the inside.

Located on each pair of straps 28 is an attachment device 36. Corresponding attachment devices 38 may be formed on exterior surfaces of the inner support member 24C and/or outer support member 24D. This may allow the straps 28 to be attached and held in a crossing pattern to the inner support member 24C and outer support member 24D of the ankle support 22. The attachment devices 36 may allow the user to adjust the tension and hence the force applied. Thus, the attachment devices 36 and 38 may allow the user to adjust and hold the straps 28 to exert more force either laterally, to counteract the foot 30 going in a supinatory movement outward and to the outside, or medially to counteract a pronatory movement inward and to the inside. The attachment devices 36 and 38 may be snaps, male/female connectors, or similar attachment devices. In accordance with one embodiment, the attachment devices 36 and 38 may be hook and loop material 36A and 38A.

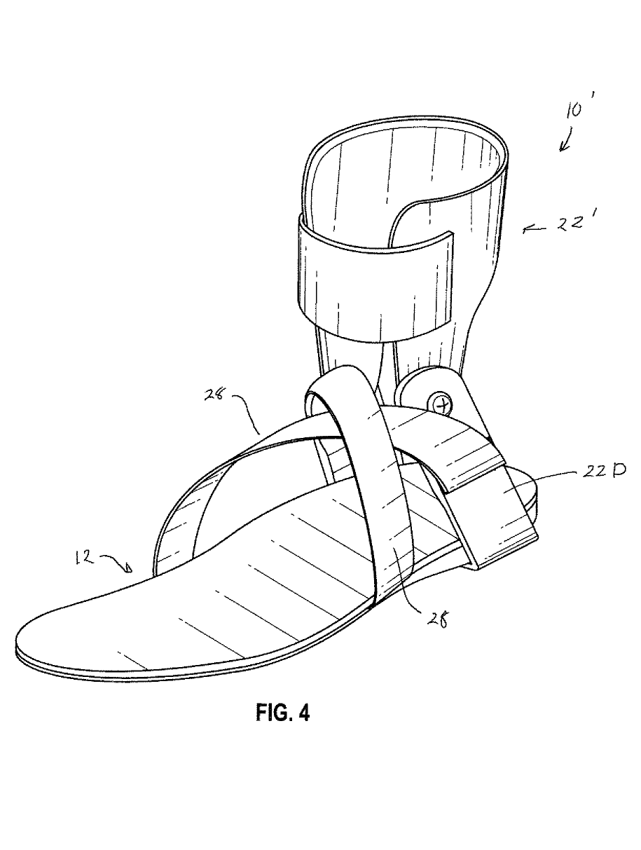

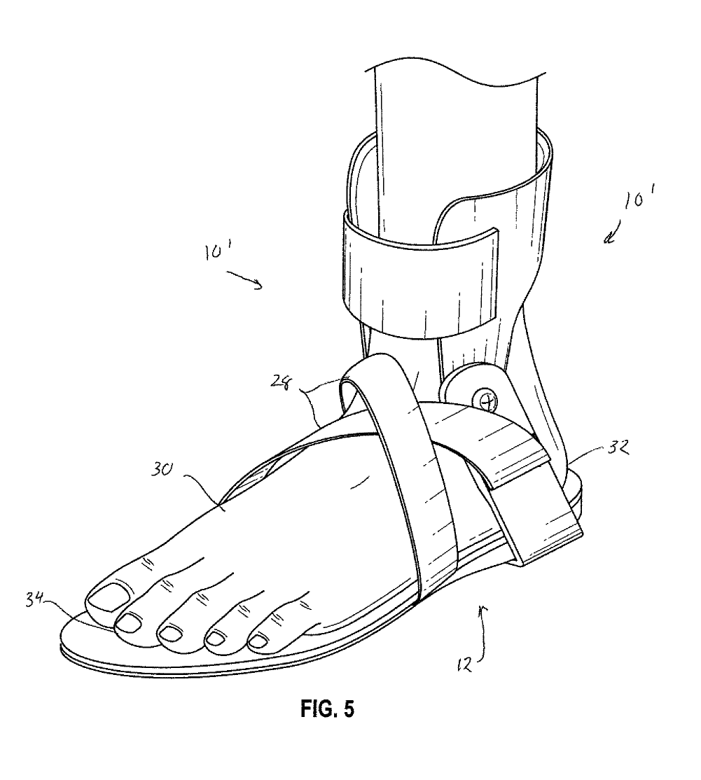

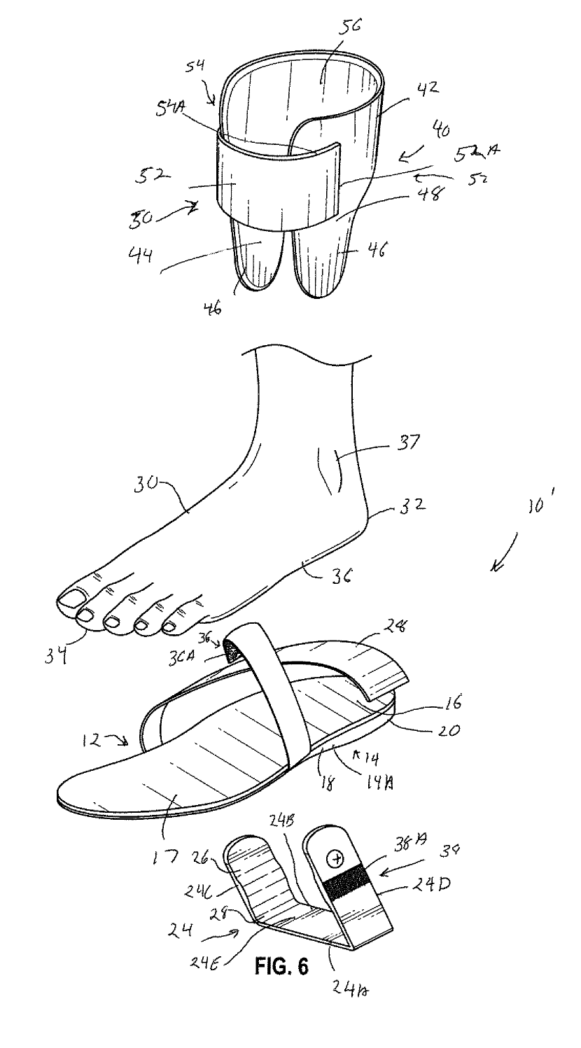

Referring now to FIG. 4-FIG. 6 another exemplary embodiment of a foot and ankle orthopedic device 10′ (hereinafter device 10′) may be shown. The device 10′ may be formed of a customizable insole 12. The insole 12 may be customized to conform to a shape of a user's foot 30. A length of the insole 12 can be dependent on foot size and may extend from the user's heel 32 towards the front 34 of the foot 30.

The insole 12 may include a base layer 14 and an upper layer 16. The base layer 14 may extend from the back of the heel 32 of the user's foot past the arch 36 of the foot 30. The base layer 14 may be molded to conform to the bottom of the foot 30. The base layer 14 may be used to control the subtalar joint and foot biomechanics of the user. In general, the base layer 14 may be formed from a thin, firm material. For example, subortholen, polypropylene, copolymer, and carbon graphite composites may be used to form the base layer. The material forming the base layer 14 may be heated to allow the material to be malleable. Once heated, the base layer may be molded to conform to a shape of the bottom of the user's foot 30. Alternatively, the material forming the base layer 14 may be milled or formed on a 3D printer to conform to a shape of the bottom of the user's foot 30.

The base layer 14 may have a heel cup 16 formed on one end thereof. The heel cup 16 may be used to keep the foot 30 from moving side to side. The heel cup 16 may further be used to elevate one side of the heel 32 to correct foot posture and gait. A medial longitudinal arch area 18 may be formed extending from the heel cup 16 along an inner side 14A of the base layer. The medial longitudinal arch area 18 may be used to support the arch 36 of the user's foot 30 and to redistribute the weight of the user to different areas to prevent excessive pressure. The medial longitudinal arch area 18 may also correct alignment issues.

A heel section 20 may be formed on a bottom section of the base layer 14 below the heel cup 16. The heel section may slightly raise the area of the base layer 14 where the heel section is located.

The upper layer 17 may be applied over the base layer 14. The upper layer 17 may extend past the base layer 14 to the end of the user's foot 30. The upper layer 17 may be formed of a flexible material. A foam, gel or similar material may be used for the upper layer 17.

An ankle support 22′ may be removably coupled to the insole 12. The ankle support 22′ may be used to support the ankle 37 of the user. The ankle support 22′ may control the position and motion of the ankle 37 due to injuries and/or irregularities of the ankle 37. Since the ankle support 22′ is removable, this may allow the user to remove the ankle support 22′ and just use the insole 12 of the device 10 as a foot orthodic.

In accordance with one embodiment, the ankle support 22′ of the present embodiment may have support members both under and along the sides of the insole 12. The support members may be formed over both the insole 12 and the corrected cast of the patient's foot and lower leg.

The ankle support 22′ may a “U” shaped member 24. The “U” shaped member 24 may be formed of a thin, semi-firm or firm material. The material used may be similar to that of the base layer 14. For example, subortholen, polypropylene, copolymer, and carbon graphite composites may be used to form the “U” shaped member 24. However, by having the material being thinner than that of the base layer 14, the ankle support 22 may have flexibility.

The bottom 24E of the “U” shaped member may be tapered from a front area 24A to a rear area 24B. The insole 12 may slide into the “U” shaped member 24 such that the heel section 20 rest within the “U” shaped member 24. The tapered configuration of the “U” shaped member 24 may allow the insole 12 to slide into the “U” shaped member 24 and prevents the heel cup 16 from running past the “U” shaped member 24.

The “U” shaped member 24 may have an inner support member 24C and an outer support member 24D. The inner support member 24C and an outer support member 24D extend up and away from the bottom of the “U” shaped member 24. The inner support member 24C and an outer support member 24D may run from the proximal/plantar to distal/dorsal areas of the ankle. In accordance with one embodiment, the inner support member 24C may be of a different height than the outer support member 24D corresponding to a location of the ankle bone. The different profile of the inner support member 24C and the outer support member 24D server multiple purposes. First, the angle and profile of the inner support member 24C may be configured to avoid the area of the navicular tuberosity. The navicular tuberosity may be a normally-occurring boney prominence on the inside of the foot 30 just above the arch 36. Prior art ankle support designs generally have support members which rub along this area and cause swelling, redness and pain. Thus, by configuring the inner support member 24C to avoid the area of the navicular tuberosity, the ankle support 22 may avoid an area of irritation and lessen the probability of swelling, redness and pain which may be caused by other ankle support devices. Second, the thinning or increased flexibility of the ankle support 22 over the malleoli (i.e., ankle bones), helps to diminish irritation of this area. Third, the profile of the inner support member 24C and the outer support member 24D may be less bulky and noticeable, thereby making it more aesthetically pleasing than prior art designs and may provide for a better shoe fit.

Padding 26 may be applied to an inner surface of the inner support member 24C and/or outer support member 24D. The padding 26 may be used to provide a more comfortable feel and to lessen irritation of the area upon which the inner support member 24C and/or outer support member 24D. A bottom interior area of inner support member 24C and/or outer support member 24D may have no padding 26. This may form a channel 28 in the bottom interior area of inner support member 24A and/or outer support member 24B. The channel 28 may provide a guide for sliding the insole 12 into the “U” shaped member 24.

A leg attachment member 40 may be coupled to the inner support member 24C and outer support member 24D. In accordance with one embodiment, the leg attachment member 40 may be movably coupled to the inner support member 24C and outer support member 24D. The leg attachment member 40 may be coupled to the inner support member 24C and outer support member 24D. In accordance with one embodiment, the leg attachment member 40 may be rotateably coupled to the inner support member 24C and outer support member 24D allowing the leg attachment member to rotate about the inner support member 24C and outer support member 24D. Alternatively, the leg attachment member 40 may be coupled in a fixed manner to the inner support member 24C and outer support member 24D.

In accordance with one embodiment, the leg attachment member 40 may have a cuff member 42. The cuff member 42 may be “C” shaped. In accordance with one embodiment, the cuff member 42 may be conical in shape having an opening 44 running along a front section thereof. The opening 44 may allow the user to insert an ankle/calf/leg of the user into the leg attachment member 40.

A pair of support arms 46 may extend down from the cuff member 42. Each of the support arms 46 may be rotatably coupled to a respective one of the inner support member 24C or outer support member 24D. Each support arm 46 may have an indented area 48. The indented area 48 may be formed in an area of the support arm 46 where the ankle bone may be located when the leg attachment member 40 is placed on a user's ankle/calf/leg. The indented area 48 may ease discomfort which may be caused by the ankle bone rubbing on the support arms 46. It should be noted that other indented areas 48 may be formed to ease discomfort which may be caused by other types of joints or protrusions rubbing on the support arms 46.

A securing device 50 may be formed across the opening 44 of the cuff member 42. The securing device 50 may be used to secure the ankle/calf/leg of the user within the cuff member 42. In accordance with on embodiment, the securing device 50 may be a strap 52. A first end of the strap 52 may be coupled to the cuff member 42 on a first side of the opening 44. A second end of the strap 52 may be removably coupled to the cuff member 42 on a second side of the opening 44, wherein the first side of the opening 44 is opposing and across from the second side of the opening 44. An attachment device 52 may be coupled to the second end of the strap 52. A corresponding attachment device 54 may be foamed on an exterior surface of the cuff member 42 on the second side of the opening 44, wherein the first side of the opening 44 is opposing and across from the second side of the opening 44. This may allow the strap 52 to be attached and held to the exterior surface of the cuff member 42 on the second side of the opening 44. The attachment devices 52 and 54 may allow the user to adjust the tension of the strap 52 across the ankle/calf/leg of the user. The attachment devices 52 and 54 may be snaps, male/female connectors, or similar attachment devices. In accordance with one embodiment, the attachment devices 52 and 54 are hook and loop material 52A and 54A.

Padding 56 may be applied on an interior surface of the cuff member 42 and the support arms 46. The padding 56 may be used to provide a more comfortable feel and to lessen irritation of the area upon which the cuff member 42 and the support arms 46 rest.

The device 10′ may have a pair of straps 28. Each strap 28 is attached to the insole 12. In accordance with one embodiment, a strap 28 is attached to opposing sides of the base layer 14 past the medial longitudinal arch area 18. The pair of straps 28 may be aligned across from each other. In accordance with one embodiment, a single strap 28 may be used. The base layer 14 may be positioned and attached to a middle area of the strap 28, opposing end members of the strap 28 forming the pair of straps 28 on opposing sides of the insole 12.

The straps 28 may be used to exert force to counteract movement of the foot 30. The straps 28 may be attached in a crossing pattern such that each strap 28 attaches to an opposing side of the ankle support 22. In accordance with one embodiment, the straps 28 may be attached in a crossing pattern to the inner support member 24C and outer support member 24D of the ankle support 22. The straps 28 may be attached in a crossing pattern over a top of the foot 30 and/or shoe of the user. The straps 28 may allow the user to exert more force either laterally, to counteract the foot 30 going in a supinatory movement outward and to the outside, or medially to counteract a pronatory movement inward and to the inside.

Located on each pair of straps 28 is an attachment device 36. Corresponding attachment devices 38 may be formed on exterior surfaces of the inner support member 24C and/or outer support member 24D. This may allow the straps 28 to be attached and held in a crossing pattern to the inner support member 24C and outer support member 24D of the ankle support 22. The attachment devices 36 may allow the user to adjust the tension and hence the force applied. Thus, the attachment devices 36 may allow the user to adjust and hold the straps 28 to exert more force either laterally, to counteract the foot 30 going in a supinatory movement outward and to the outside, or medially to counteract a pronatory movement inward and to the inside. The attachment device 36 may be snaps, male/female connectors, or similar attachment devices. In accordance with one embodiment, the attachment device 36 is hook and loop material 36A.

The foregoing description is illustrative of particular embodiments of the invention, but is not meant to be a limitation upon the practice thereof. The following claims, including all equivalents thereof, are intended to define the scope of the invention.

What is claimed is:

1. An ankle and foot orthotic device comprising:
an insole; and
an ankle support positioned under the insole and extending up above a foot and proximate an ankle of a user.

2. The ankle and foot orthotic device of claim 1 comprising a pair of straps, wherein an individual strap is coupled to opposing sides of the insole, wherein the pair of straps arranged in a crossing pattern to exert force to counteract movement of the foot.

3. The ankle and foot orthotic device of claim 1, wherein the pair of straps arranged in a crossing pattern to exert force one of laterally to counteract the foot going in a supinatory movement outward or medially to counteract a pronatory movement inward.

4. The ankle and foot orthotic device of claim 1, wherein the ankle support is removable allowing the insole to be used separately.

5. The ankle and foot orthotic device of claim 1, wherein the insole comprises:
a base layer extending from a back of a heel of the foot past an arch of the foot;
a heel cup formed on one end of the base layer;
a medial longitudinal arch area extending from the heel cup along an inner side of the base layer; and
an upper layer applied over the base layer, the upper layer 16 extending past the base layer to an end of the foot.

6. The ankle and foot orthotic device of claim 5, wherein the insole comprises a heel section formed on a bottom section of the base layer below the heel cup.

7. The ankle and foot orthotic device of claim 5, wherein the upper layer is formed of a flexible material.

8. The ankle and foot orthotic device of claim 1, wherein the ankle support comprises:
a “U” shaped member,
an inner support member extending up and away from the bottom of the “U” shaped member;
an outer support member extending up and away from the bottom of the “U” shaped member;
wherein the inner support member and the outer support member run from the proximal/plantar to distal/dorsal areas of the ankle.

9. The ankle and foot orthotic device of claim 8, wherein the inner support member and the outer support member are semi-flexible to reduce irritation.

10. The ankle and foot orthotic device of claim 8, wherein a bottom area of the “U” shaped member is tapered from a front area to a rear area, the insole sliding into the “U” shaped member.

11. The ankle and foot orthotic device of claim 8, wherein the ankle support comprises a leg attachment member rotatably coupled to the inner support member and outer support member.

12. The ankle and foot orthotic device of claim 11, wherein the leg attachment member comprises:
a “C” shaped cuff member, the cuff member being conical in shape;
an opening running along a front section of the “C” shaped cuff member;
a pair of support arms extending down from the cuff member, each of the support arm rotatably coupled to a respective one of the inner support member or outer support member.

13. The ankle and foot orthotic device of claim 12, wherein the pair of support arms each has an indented area formed in an area of each support arm where an ankle bone is located.

14. The ankle and foot orthotic device of claim 12, comprising a securing device formed across the opening of the cuff member.

15. An ankle and foot orthotic device comprising:
an insole;
an ankle support positioned under the insole and extending up above a foot and proximate an ankle of a user, wherein the ankle support comprises:
a “U” shaped member;
an inner support member extending up and away from the bottom of the “U” shaped member; and
an outer support member extending up and away from the bottom of the “U” shaped member;
wherein the inner support member and the outer support member run from the proximal/plantar to distal/dorsal areas of the ankle;
wherein the inner support member and the outer support member are semi-flexible to reduce irritation and
a pair of straps, wherein an individual strap is coupled to opposing sides of the insole, wherein the pair of straps arranged in a crossing pattern and coupled to a respective one of the inner support member and outer support member to exert force to counteract movement of the foot.

16. The ankle and foot orthotic device of claim 15, wherein the pair of straps arranged in a crossing pattern to exert force one of laterally to counteract the foot going in a supinatory movement outward or medially to counteract a pronatory movement inward.

17. The ankle and foot orthotic device of claim 15, wherein a bottom area of the “U” shaped member is tapered from a front area to a rear area, the insole sliding into the “U” shaped member.

18. The ankle and foot orthotic device of claim 15, wherein the insole comprises:
a base layer extending from a back of a heel of the foot past an arch of the foot;
a heel cup formed on one end of the base layer;
a medial longitudinal arch area extending from the heel cup along an inner side of the base layer;
an upper layer applied over the base layer, the upper layer 16 extending past the base layer to an end of the foot; and
a heel section formed on a bottom section of the base layer below the heel cup, wherein heel section slides into the “U” shaped member.

19. The ankle and foot orthotic device of claim 15, wherein the ankle support comprises:
a “C” shaped cuff member, the cuff member being conical in shape;
an opening running along a front section of the “C” shaped cuff member;
a pair of support arms extending down from the cuff member, each of the support arm rotatably coupled to a respective one of the inner support member or outer support member, wherein the pair of support arms each has an indented area formed in an area of each support arm where an ankle bone is located; and
a securing device formed across the opening of the cuff member.

20. An ankle and foot orthotic device comprising:
an insole, wherein the insole comprises:
a base layer extending from a back of a heel of the foot past an arch of the foot;
a heel cup formed on one end of the base layer;
a medial longitudinal arch area extending from the heel cup along an inner side of the base layer;
an upper layer applied over the base layer, the upper layer 16 extending past the base layer to an end of the foot; and
a heel section formed on a bottom section of the base layer below the heel cup, wherein heel section slides into the “U” shaped member;
an ankle support positioned under the insole and extending up above a foot and proximate an ankle of a user, wherein the ankle support comprises:
a “U” shaped member, the “U” shaped member being tapered from a front area to a rear area, the heel section sliding into and held with the “U” shaped member;
an inner support member, the outer support member extends up and away from the bottom of the “U” shaped member; and
an outer support member, the outer support member extends up and away from the bottom of the “U” shaped member;
wherein the inner support member and the outer support member run from the proximal/plantar to distal/dorsal areas of the ankle;
wherein the inner support member and the outer support member are semi-flexible to reduce irritation; and
a pair of straps, wherein an individual strap is coupled to opposing sides of the insole, wherein the pair of straps arranged in a crossing pattern to exert force one of laterally to counteract the foot going in a supinatory movement outward or medially to counteract a pronatory movement inward.

21. The ankle and foot orthotic device of claim 20, wherein the ankle support comprises:
a “C” shaped cuff member, the cuff member being conical in shape;
an opening running along a front section of the “C” shaped cuff member;
a pair of support arms extending down from the cuff member, each of the support arm rotatably coupled to a respective one of the inner support member or outer support member, wherein the pair of support arms each has an indented area formed in an area of each support arm where an ankle bone is located; and
a securing device formed across the opening of the cuff member.
